(19) United States
(12) Patent Application Publication
(10) Pub. No.: US 2010/0010419 A1
(43) Pub. Date: Jan. 14, 2010

(54) Phacoemulssification Needle Tips

(76) Inventor: Takayuki Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 12/501,825

(22) Filed: Jul. 13, 2009

Related U.S. Application Data
(60) Provisional application No. 61/080,681, filed on Jul. 14, 2008.

(51) Int. Cl. A61F 9/007

(52) U.S. Cl. 604/22

(57) ABSTRACT

A phacoemulsification needle has tip geometries configured to operate efficiently in both the longitudinal and torsional modes. In one variation, the needle tip has a sharpened edge formed on at least a portion of the needle lip. In other variations the lip geopmetry is selected to create cutting surfaces in both the torsional and longitudinal directions.

### PRIORITY

This application claims priority from U.S. Patent Application Ser. No. 61/080,681, filed Jul. 14, 2008, entitled “Phacoemulsification Needle Tips”, which is incorporated herein in its entirety by reference.

### FIELD OF THE INVENTION

This disclosure relates to surgical instruments and surgical techniques used in eye surgery and more particularly, to phacoemulsification apparatus and methods for their use.

### BACKGROUND OF THE INVENTION

A common ophthalmological surgical technique is the removal of a diseased or injured lens from the eye. Earlier techniques used for the removal of the lens typically required a substantial incision to be made in the capsular bag in which the lens is encased. Such incisions were often on the order of 12 mm in length.

Later techniques focused on removing diseased lenses and inserting replacement artificial lenses through as small an incision as possible. For example, it is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece to which is attached a hollow needle. Electrical energy is applied to vibrate the needle at ultrasonic frequencies in order to fragment the diseased lens into small enough particles to be aspirated from the eye through the hollow needle. Commonly, an infusion sleeve is mounted around the needle to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small in incision as possible during such surgery is the minimization of leakage of liquid during and after surgery and the prevention of such a collapse.

Phacoemulsification needles and tips are well represented in the prior art. Needles and tips of varying configurations are well known. A particular shape for a tip or needle is often dictated by the type of handpiece with which the needle is to be used.

U.S. Pat. No. 5,788,679 (Gravlee, Jr.) teaches and describes a phacoemulsification needle having a sharpened edge formed at the tip by cutting the needle tip at an angle to form bevels. The resulting needle is not a straight needle and the needle must be held at an angle to bring portions of the cutting edge into contact with the nucleus or other tissue to be treated. This needle configuration appears to be suitable for a handpiece using linear or longitudinal motion but not for one using torsional motion.

United States Patent Application Publication 2006/0217672 (Chon) teaches and describes a phacoemulsification tip that is swaged or crimped at its distal end. The tip is intended for use with a handpiece producing torsional motion and the crimping forms cutting edges at the distal end.

U.S. Pat. No. 5,725,495 (Strukel et al) teaches and describes a phacoemulsification handpiece, sleeve and tip illustrating a wide variety of tip configurations and needle cross-sectional configurations.

U.S. Pat. No. 6,007,555 (Devine) teaches and describes an ultrasonic needle for surgical emulsification. The needle and its tip are shown in both circular and oval configurations.

U.S. Pat. No. 6,605,054 (Rockley) teaches and describes a multiple bypass port phaco tip having multiple aspiration ports and a single discharge port to infuse liquid into the eye.

U.S. Pat. No. 5,879,356 (Geuder) teaches and describes a surgical instrument for crushing crystalline eye lenses by means of ultrasound and for removing lens debris by suction which demonstrates the use of a sleeve positioned concentric to the needle and having a pair of discharge ports formed thereon.

U.S. Pat. No. 5,645,530 (Boukhny) teaches and describes a phacoemulsification sleeve, one variation of which has a bellows portion attached to a discharge port ring which directs an annular flow of liquid around the needle and into the eye. The use of the bellows is intended to allow the sleeve to absorb spikes in liquid pressure during the operation.

Published United States Patent Application No. 2003/0004455 (Kadziauskas) teaches and describes a bi-manual phaco needle using separate emulsification and aspiration needles inserted into the eye simultaneously during surgery..

U.S. Pat. No. 6,077,285 (Boukhny) teaches and describes a torsional ultrasound handpiece configured to impart both longitudinal and torsional motion to a phacoemulsification needle.

U.S. Pat. No. 6,402,769 (Boukhny) is a continuation in part of the '285 patent and further particularizes the frequencies at which the crystals providing both the torsional and longitudinal motion are activated.

I have determined that improved results can be achieved if the lip of a straight phacoemulsification needle tip is formed in a variety of differing configurations. In particular, certain of these configurations provide lips with varying degrees of sharpness for use in different phaco procedures. I have also determined that the selected configurations can also be used with angled tips. I have also determined that varying configurations provide advantages for phacoemulsification needles used with both linear and torsional hand pieces. Such tips are intended to be of particular value when used with handpieces capable of generating both longitudinal and torsional motion, or a blend of such motions.

In accordance with these criteria, I have designed a series of phacoemulsification needle tips having shaped lips whose configuration is selected by the task to be performed.

While the following describes an example or examples of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### BRIEF DESCRIPTION OF THE DRAWINGS

These and further aspects of the present invention will be best understood by reference to the accompanying drawings wherein:

FIG. 1 is a drawing showing prior art oval and square-shaped tips;

FIG. 2 is a drawing showing several prior art needle cross-sectional configurations;

FIG. 3 is a lateral sectional view of a prior art needle tip;

FIG. 4 is a perspective view of the needle tip of FIG. 3;

FIG. 5 is a lateral schematic sectional view of a prior art needle tip;

FIG. 6 is an enlarged view of the tip of FIG. 5;

FIG. 7 is a schematic view of the tip of FIG. 5 used with a longitudinal hand piece;

FIG. 8 is a schematic view of the tip of FIG. 5 used with a torsional hand piece;

FIG. 9 is a partial schematic sectional view of a tip embodying certain principles of the present invention;

FIG. 10 is an enlarged view of the portion of the tip of FIG. 9;

FIG. 11 is a schematic view of the tip of FIG. 9 used with a hand piece having longitudinal motion;

FIG. 12 is a schematic view of the tip of FIG. 9 used with a hand piece having torsional motion;

FIG. 13 is a partial schematic sectional view of another embodiment of the present invention;

FIG. 14 is an enlarged view of a portion of the tip of FIG. 13;

FIG. 15 is a schematic view of the tip of FIG. 13 used with a hand piece having longitudinal motion;

FIG. 16 is a schematic view of the tip of FIG. 13 used with a hand piece having torsional motion;

FIG. 17 is a partial schematic sectional view of another embodiment of the present invention;

FIG. 18 is an enlarged view of a portion of the tip of FIG. 17;

FIG. 19 is a schematic view of the tip of FIG. 16 used with a hand piece having longitudinal motion;

FIG. 20 is a schematic view of the tip of FIG. 17 used with a hand piece having torsional motion;

FIG. 21 is a partial schematic sectional view of another embodiment of the present invention;

FIG. 22 is an enlarged view of a portion of the tip of FIG. 21;

FIG. 23 is a schematic view of the tip of FIG. 21 used with a hand piece having longitudinal motion;

FIG. 24 is a schematic view of the tip of FIG. 21 used with a hand piece having torsional motion;

FIG. 25 is a partial schematic sectional view of another embodiment of the present invention;

FIG. 26 is an enlarged view of a portion of the tip of FIG. 26;

FIG. 27 is a partial sectional view of another embodiment of the present invention;

FIG. 28 is is a partial sectional view of another embodiment of the present invention;

FIG. 29 is a partial sectional view of another embodiment of the present invention;

FIG. 30 is a partial sectional view of another embodiment of the present invention;

FIG. 31 is a partial sectional view of another embodiment of the present invention;

FIG. 32 is a partial sectional view of another embodiment of the present invention;

FIG. 33 is a partial sectional view of another embodiment of FIG. 33; and

FIG. 34 is a partial sectional view of another embodiment of the present invention.

### DETAILED DESCRIPTION OF THE INVENTION

It is common for some in the art to refer to an entire phacoemulsification needle as a “tip”. For the purposes of the description that follows, the word “tip” shall refer to that portion of a phacoemulsification needle that is proximate the end of the needle that contacts the tissue to be emulsified. The portion of the needle extending from the tip to the needle mount shall be referred to as the “needle body”.

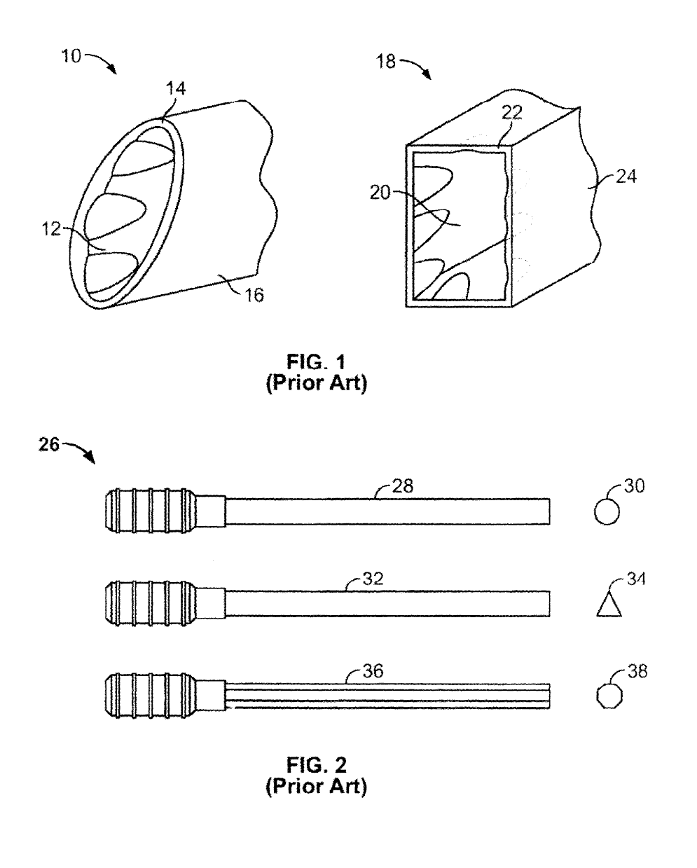

Referring now to FIG. 1, the numeral 10 indicates generally a prior art phacoemulsification needle tip as shown in U.S. Pat. No. 6,007,555. Needle 10 terminates in a mouth 12 defined by a lip 14 at the end of needle body 16, with lip 14 and needle body 16 formed as having an oval cross-section configuration.

Referring to FIG. 1, the numeral 18 indicates generally a prior art phacoemulsification needle tip from U.S. Pat. No. 6,007,555, having a mouth 20 defined by a lip 22 at the end of needle 24. The cross-sectional configuration of needle 18 and mouth 20 is a rectangle.

Referring now to FIG. 2, the numeral 26 identifies several prior art phacoemulsification needles as described in U.S. Pat. No. 5,725,495, with needle 28 having a circular cross-section as shown at 30, needle 32 having a triangular cross-section as shown at 34 and needle 36 having an octagonal cross-section as shown at 38.

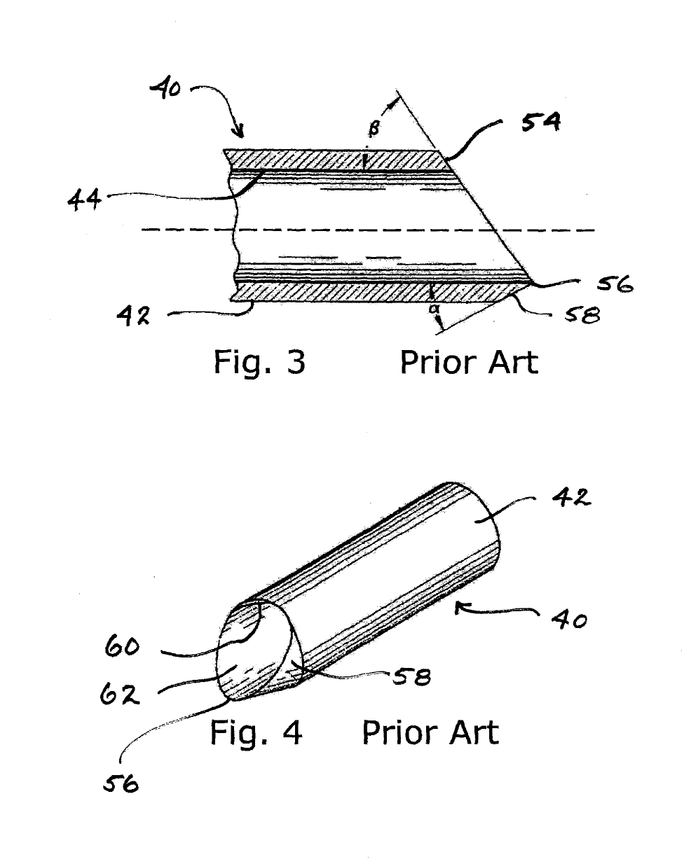

Referring now to FIG. 3, the numeral 40 indicates generally a partial sectional view of a prior art phacoemulsification needle tip shown and described in U.S. Pat. No. 5,788,679. Needle tip 40 has a side wall 42 having an inner surface 44 terminating at mouth 50. As seen in FIG. 3, mouth 50 is formed at angle β with respect to axis 52 forming a trailing edge 54 and a leading edge 56 as seen in FIG. 3. As further seen, leading edge 56 is formed with a bevel 58 formed at an angle α to inner surface 44. This appears to result in at least a portion of leading edge 56 formed with a somewhat sharpened edge. As seen in FIG. 4, the portion of edge 56 shown is the flat or blunt portion 60 of trailing edge 54. As seen in FIG. 3 edges 54 and 56 define needle mouth 62.

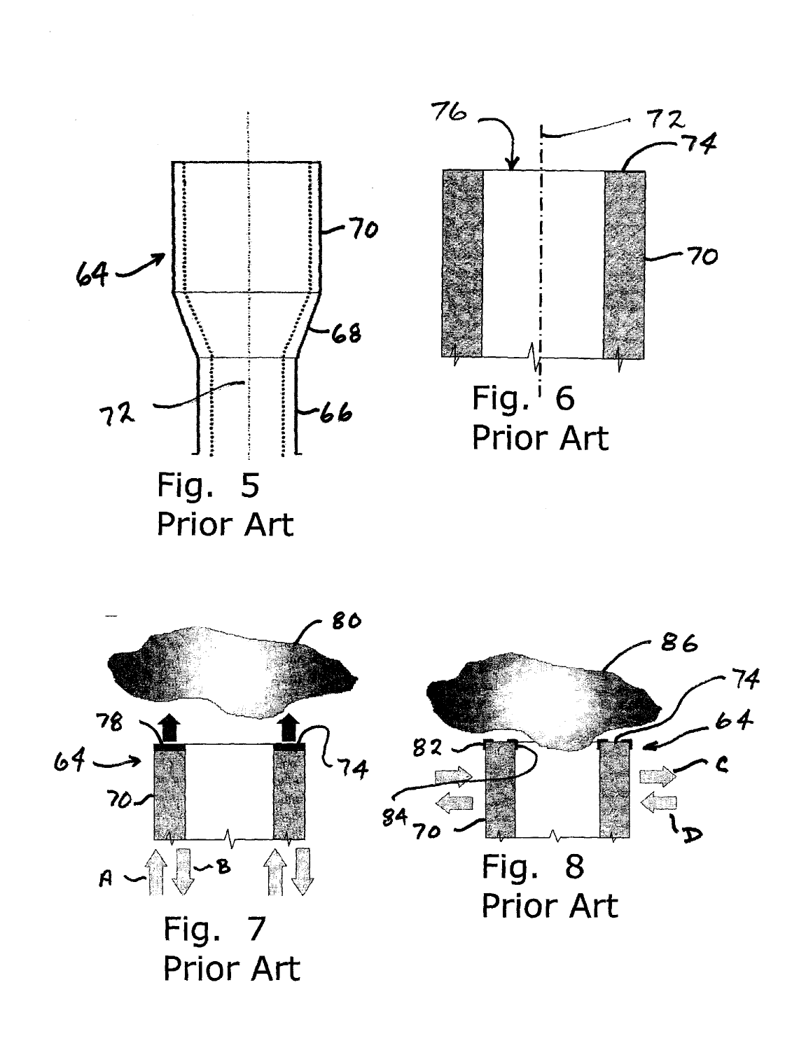

Referring now to FIG. 5, the numeral 64 identifies a prior art phacoemulsification needle tip having been formed as part of needle body 66 with an enlarged tapered section 68 terminating in a tip 70 having straight walls extending parallel to axis 72. As seen in FIG. 5 and more particularly in FIG. 6, tip portion 70 terminates at a lip 74 which defines the tip mouth 76 with lip 74 formed substantially perpendicular to axis 72.

Referring to FIG. 7, the use of tip 64 is illustrated on a hand piece providing longitudinal motion meaning that tip 70 is moved, alternatively, in the directions indicated by arrows A and B. As seen in this illustration, such motion provides tip 64 with an effective cutting zone 78 extending generally about the periphery of lip 74. In my experience, such a configuration tends to repulse nucleus 80 in a direction away from cutting zone 78 making it more difficult to apply an emulsification force to nucleus 80.

Referring now to FIG. 8, the use of tip 64 in a hand piece providing torsional motion is shown, meaning that the handpiece imparts an alternating rotational motion to a phacoemulsification needle attached to the handpiece is rotated repetitively in a clockwise direction and a counterclockwise direction imparting an eccentric or “torsional” motion to the needle tip, such as tip 64. This results in the creation of an exterior cutting zone 82 and an interior cutting zone 84 extending about the inner and outer peripheries of tip 70, a configuration which provides less efficient cutting when tip 70 is used to cut nucleus 86.

Throughout, the use of arrows A and B in an illustration will identify movement of the tip in the longitudinal direction by the handpiece to which the phacoemulsification needle is mounted. Throughout, the use of arrows C and D in an illustration will identify movement of the tip in the torsional direction by the handpiece to which the phacoemulsification needle is mounted.

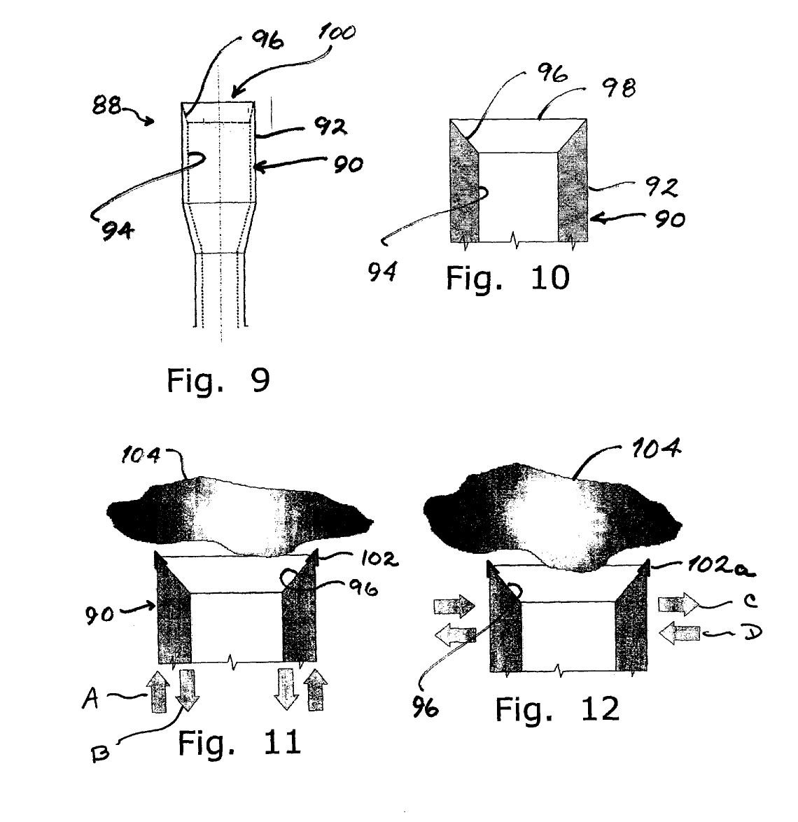

Referring now to FIG. 9, the numeral 88 identifies a tip configuration embodying certain of the principles of the present invention. Needle 88 terminates in a tip 90 having an outer wall 92 and an inner wall 94 which, in turn, terminates at a lip 96. As seen in greater detail in FIG. 10, lip 96 is formed at an angle to outer wall 92 to form a sharpened and continuous tip edge 98 surrounding tip mouth 100.

As seen in FIG. 11, the configuration of tip 90 creates an effective cutting zone 102 extending about the outer periphery of lip 96 when tip 90 is used in a longitudinal motion. Cutting zone 102 is believed to extend inwardly along a portion of lip 96 and along a portion of the outer surface 92 extending downwardly from edge 98. It is expected that such a configuration will result in diminished repulsion of nucleus 104 during the phacoemulsification process.

Referring now to FIG. 12, tip 90 is shown used with a handpiece having torsional motion. It is expected that tip 90, when used with a hand piece having torsional motion will create an effective cutting zone 102a which will increase the efficiency of tip 90 when cutting nucleus 104.

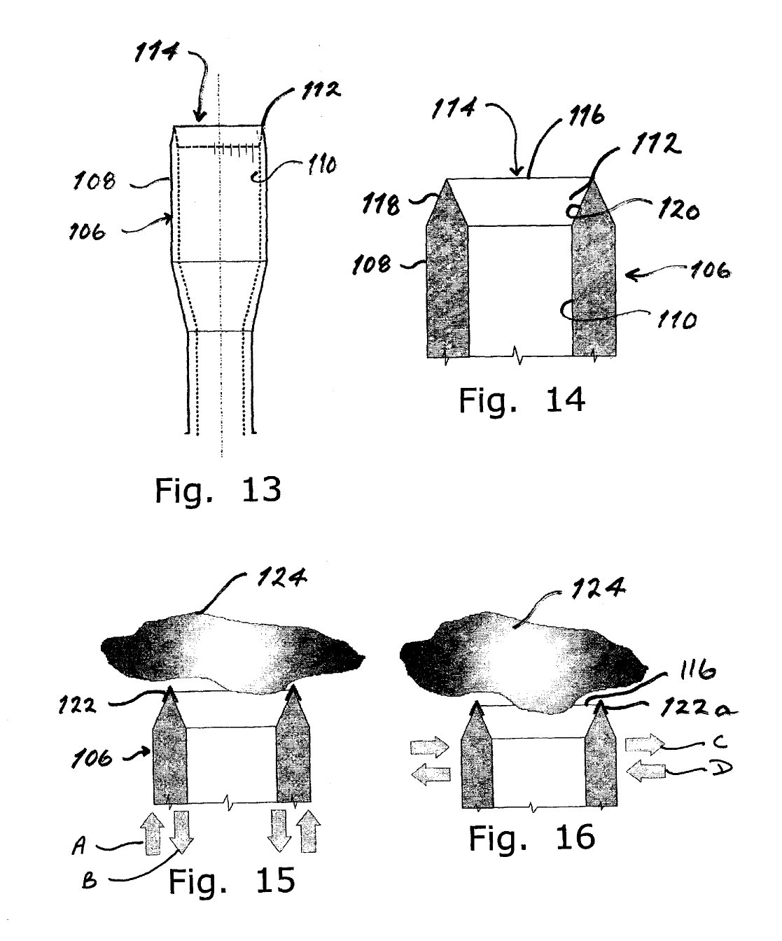

Referring now to FIGS. 13 and 14, another embodiment of the present invention is shown with needle tip 106 having an outer wall 108 and an inner wall 110 terminating at a lip 112 and defining a tip mouth 114.

As seen in FIG. 14, tip 112 terminates in a cutting edge 116 formed by beveling a first portion 118 of outer surface 108 and a second portion 120 of inner surface 110 to intersect and form cutting edge 116. Tip 106 therefore terminates in an outer beveled surface 188 and an inner beveled surface 120.

As seen in FIG. 15, it is expected that tip 106 will create an effective cutting zone 122 when used with a hand piece having longitudinal motion and that such a configuration will reduce the tendency of nucleus 124 to be repulsed by the motion of tip 106.

Referring now to FIG. 16, tip 106 is shown as used with a hand piece having torsional motion and it is expected that the configuration of tip 106 will create an effective cutting zone 122a along edge 116 and will result in more efficient phacoemulsification of nucleus 124.

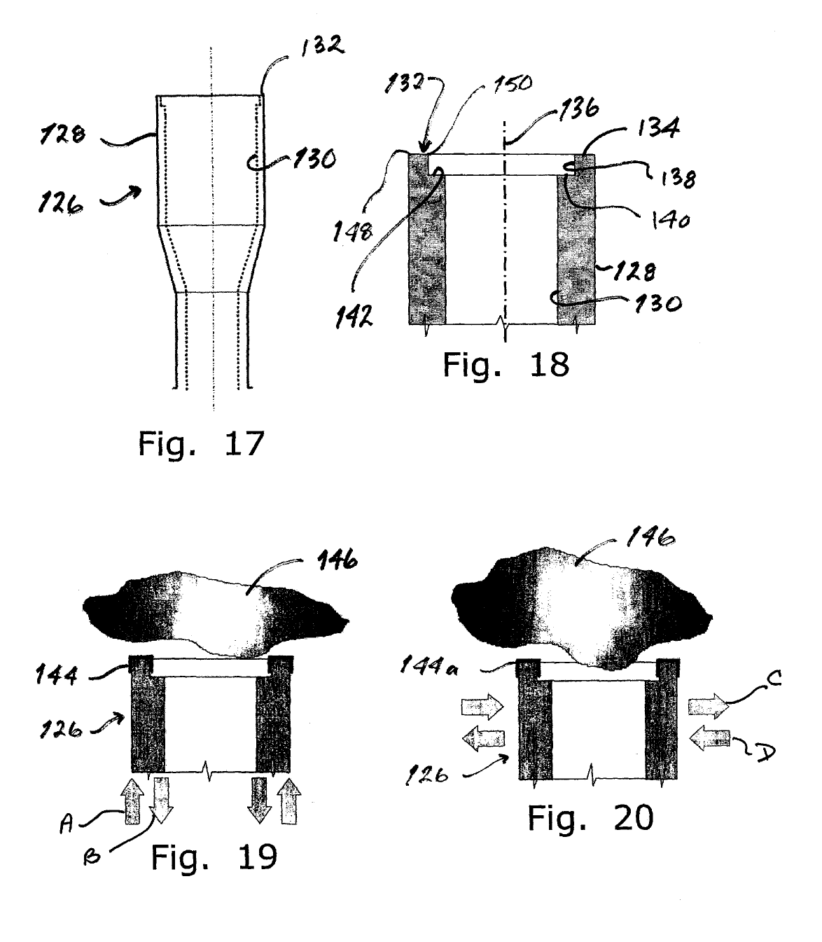

Referring to FIG. 17 another embodiment of the present invention is illustrated with tip 126 having an outer wall 128, and an inner wall 130 terminating at lip 132. As seen in greater detail in FIG. 18, lip 132 is formed with a step-like configuration having an outer lip portion 134 extending generally perpendicular to axis 136 and terminating intermediate outer wall 128 and inner wall 130. Tip 132 is further characterized by an inner wall portion 138 extending downward in a direction generally parallel to axis 136. Wall portion 138 terminates at a point distal from tip portion 134 at an inwardly-extending wall portion 140 in a direction generally toward and perpendicular to axis 136, thereby forming tip land 142, which is defined by wall portions 138, 140 and which extends between wall portion 138 and inner wall 130. In this embodiment, land 142 is formed generally or substantially perpendicular to axis 136. It should be understood that the angle of wall portion 140 and land 142 with respect to 136 may be altered to create different cutting characteristics for tip 126.

Referring now to FIG. 19, the use of tip 126 is shown with a hand piece having longitudinal motion. It is expected that tip 126 will create an effective cutting zone 144 which will reduce the tendency of tip 126 to repel nucleus 146 thus resulting in more efficient phacoemulsification. It is expected that the creating of cutting zone 144 results from the motion of outer tip edge 148 and inner tip edge 150 as shown in FIG. 19.

Referring now to FIG. 20, use of tip 126 is shown with a hand piece having torsional motion. It is expected that this configuration will create and effective cutting zone 144a which will increase the efficiency of the cutting of nucleus 146.

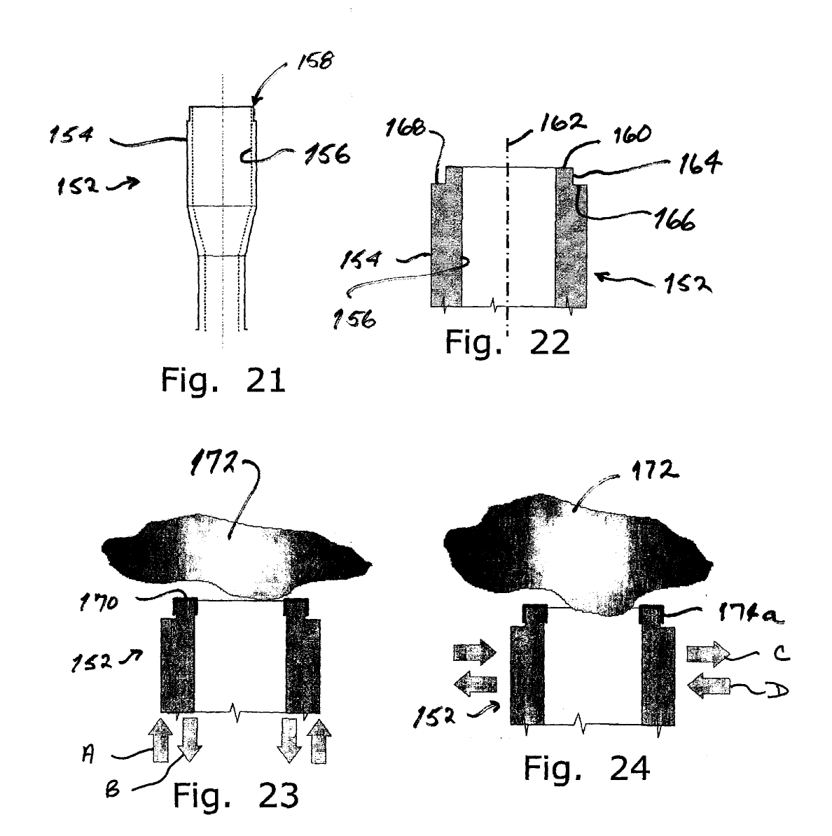

Referring to FIG. 21 another embodiment of the present invention is illustrated with tip 152 having an outer wall 154, and an inner wall 156 terminating at lip 158. As seen in greater detail in FIG. 22, lip 158 is formed with a step-like configuration having an outer lip portion 160 extending generally perpendicular to axis 162 and intermediate outer wall 154 and inner wall 156. Tip 132 is further characterized by an outer lip portion 164 extending downward in a direction generally parallel to axis 162. Outer lip portion 164 terminates in a point distal from lip 158 to form an outwardly-extending wall portion 166 in a direction generally perpendicular to axis 164, thereby forming external tip land 168, which is defined by wall portions 164, 166 and which extends between outer lip portion 164 and outer wall 154. In this embodiment, land 168 is formed generally or substantially perpendicular to axis 162. It should be understood that the angle of outer lip portion 164 and land 168 with respect to axis 162 may also be altered to create different cutting characteristics for tip 152.

Referring now to FIG. 23, the use of tip 152 is shown with a hand piece having longitudinal motion. It is expected that tip 152 will create an effective cutting zone 170 which will reduce the tendency of tip 152 to repel nucleus 172 thus resulting in more efficient phacoemulsification. It is expected that the creating of cutting zone 170 results from the formation of outer lip portion 160 and outwardly-extending wall portion 166 as shown in both FIGS. 22 and 23.

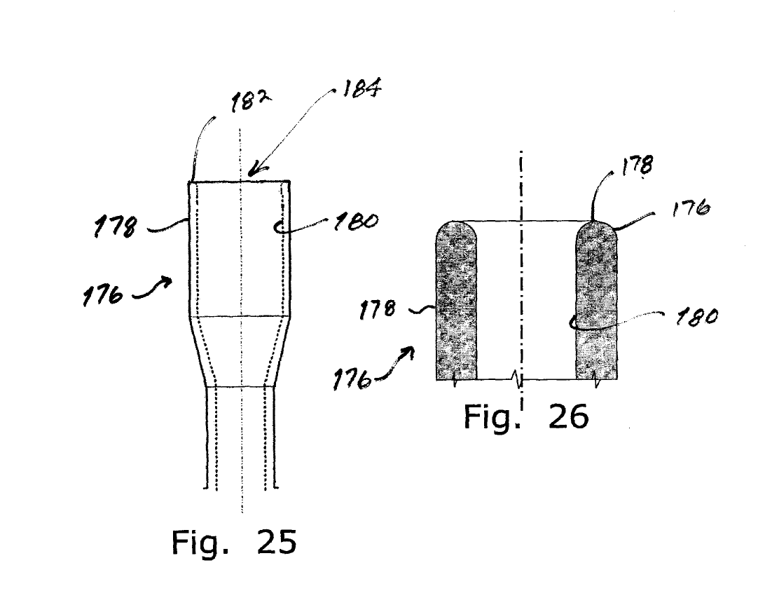

Referring now to FIG. 24, use of tip 152 is shown with a hand piece having torsional motion. It is expected that this configuration will create and effective cutting zone 174a which will increase the efficiency of the cutting of nucleus 172.

Referring now to FIG. 25, another embodiment of the present invention is illustrated by needle tip 176 having outer surface 178 and inner surface 180 with said inner and outer surfaces terminating at lip 182 defining a mouth 184.

Referring now to FIG. 26, tip 176 is shown in greater detail. Lip 176 is shown having a radiused cross-sectional shape formed by a curvilinear surface 178 extending from outer surface 178 to inner surface 180. This configuration provides a single, continuous, relatively smooth and blunt cutting surface which, it is expected, will be more desirable for surgical situations in which relatively thin or sensitive tissue, such as the posterior capsule, may be contacted during emulsification. The added protection is useful with both longitudinal and torsional motion.

The foregoing examples have shown straight tip configurations, that is, where the lip defining the mouth lies generally in a plane perpendicular to the tip's longitudinal axis. It is also expected that the foregoing tip configurations will be effective if the lip is angled with respect to the longitudinal axis and the tip configurations described above are formed thereon.

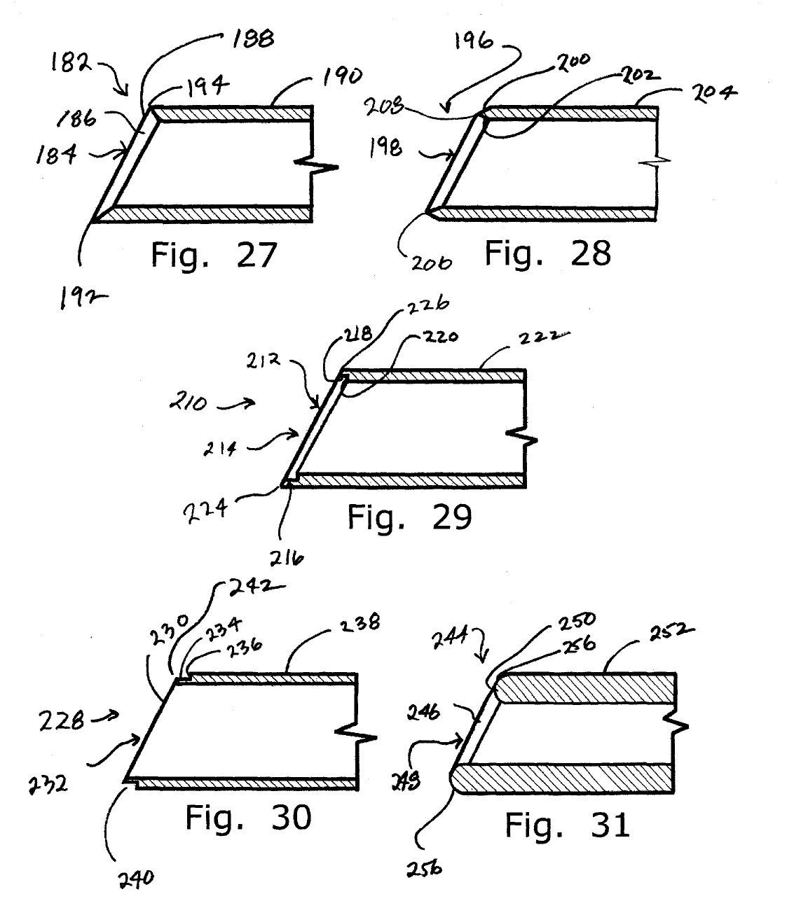

Referring now to FIG. 27, the numeral 182 identifies a tip having an angled lip 184 configured substantially as shown in FIGS. 9-12. Lip 184 defines a mouth 186. Cutting edge 188 corresponds to cutting edge 98 in FIG. 10. As seen, lip 184 is beveled with respect to tip wall 190, creating a leading edge 192 and a trailing edge 194 of cutting edge 188.

Referring now to FIG. 28, the numeral 196 identifies a tip having an angled lip 198 configured substantially as shown in FIGS. 13-16. Lip 198 is formed by tapered wall segments 200, 202 of tip wall 204. As seen, lip 198 is angled with respect to tip wall 204 creating a leading edge 206 and a trailing edge 208.

Referring now to FIG. 29, the numeral 210 identifies a tip having an angled lip configured substantially as shown in FIGS. 17-20. Tip 210 has a lip 212 defining a mouth 214. Lip 212 is formed with an internal step configuration 216 having a first axial wall segment 218 and a second radial wall segment 220 forming a step with a configuration such as that shown in FIG. 18. As seen in FIG. 29, lip 212 is beveled or angled with respect to tip wall 222 forming a leading edge 224 and a trailing edge 226.

Referring now to FIG. 30, the numeral 228 identifies a tip configured substantially as shown in FIGS. 21-24. Tip 228 has a lip 230 which defines mouth 232 and which has a first, axially extending segment 234 formed proximate lip 230 and a second, generally radially extending segment 236 contiguous to segment 234 to form the external steps as seen in FIG. 22. As seen in FIG. 30, lip 230 is angled with respect to tip wall 238 creating a leading edge 240 and a trailing edge 242.

Referring now to FIG. 31, the numeral 244 identifies an angled tip configured substantially as shown in FIGS. 26-27, having an angled lip 246 defining a mouth 248. As seen in FIG. 31, lip 246 is rounded at its endmost portion 250 in the manner shown in FIG. 26. Lip 246 is also angled with respect to tip wall 252 to form an angled configuration with a leading edge 254 and a trailing edge 256.

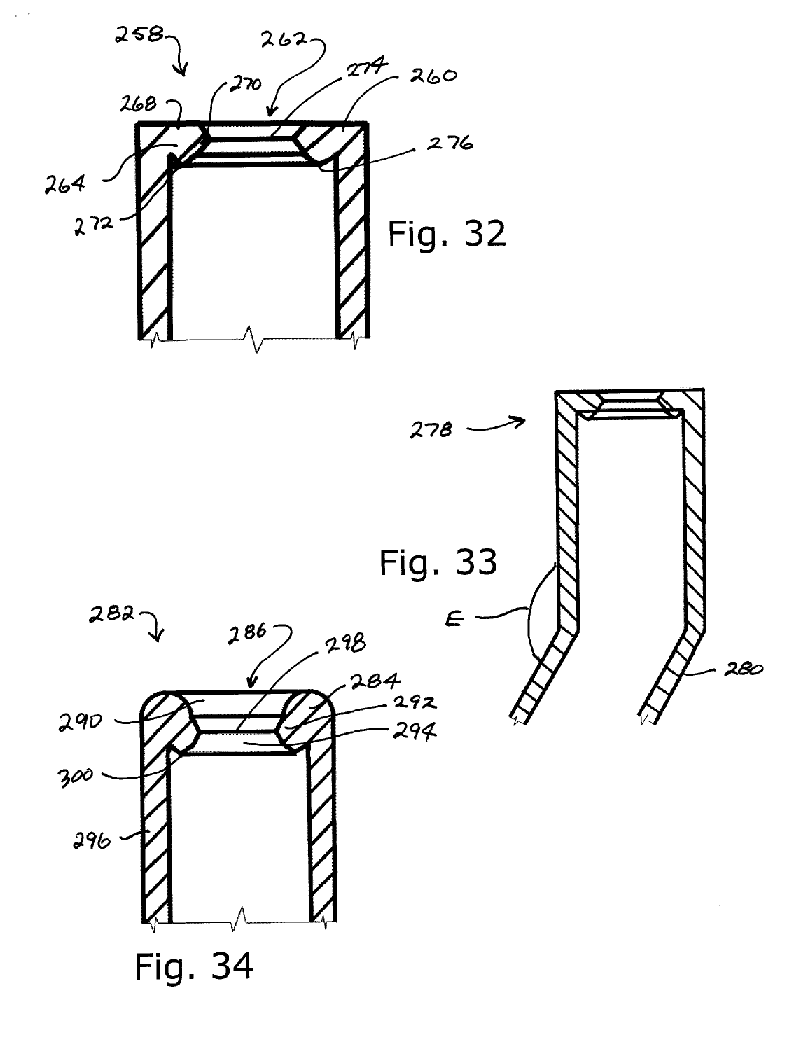

Referring now to FIG. 32 the numeral 258 identifies a tip having a lip 260 defining a mouth 262. Lip 260 has a lip segment 264 extending from tip wall 266 partially into mouth 262. Lip segment 264 has a first, radial lip surface 268 extending into mouth 262, a second, axial lip surface 270 depending from surface 266 and a third, radial surface 272 extending from surface 268 to tip wall 266.

In the embodiment shown, axial surface 270 is beveled to create an edge 274 which acts as a cutting edge. In like fashion, third lip surface 272 may be flat or may be beveled to form an edge 276, each of which acts as a cutting surface.

Referring now to FIG. 33 the numeral 278 identifies a tip constructed in accordance with the description of tip 262 shown in FIG. 32. In this embodiment, tip 278 is formed at an angle E to needle body 280.

Referring now to FIG. 34, a tip 282 is shown in partial cross-section, having a lip 284 defining a mouth 286. Lip 284 has a lip segment 288 extending from tip wall 290 partially into mouth 286. Lip segment 288 has a first, radial lip surface 290 extending into mouth 286, a second, axial lip surface 292 depending from surface 290 and a third, radial surface 294 extending from surface 292 to tip wall 296.

In the embodiment shown, first lip surface 290 is formed with a smoothed, rounded or polished surface, while axial surface 292 is beveled to create an edge 298 which acts as a cutting edge. In like fashion, third lip surface 294 is beveled to form an edge 300 which acts as a cutting edge. First lip surface 290 helps protect delicate eye tissue from damage during phacoemulsification.

The foregoing descriptions are made with the understanding that the configuration of the tip is flared, that is, the tip is larger than the phacoemulsification needle body. It is expected that straight phacoemulsification needles having no enlargement or flare at the tip will also benefit from the application of the principles of the present invention.

The foregoing descriptions are also made with the understanding that the tips may be either straight or angled with respect to the needle body and can be used with handpieces having either longitudinal or torsional motion, and are expected to be more efficient when the tips are angled with respect to the needle body.

What is claimed is:

1. A phacoemulsification needle for use with a handpiece capable of imparting to said needle a longitudinal motion or a torsional motion, said needle comprising:
a needle body having a proximal end and a distal end,
said needle body having a central passageway;
a needle mount formed at said proximal end,
said needle mount adapted to attach said needle to said handpiece;
a needle tip formed at said distal end,
said needle tip having an inner surface and an outer surface;
said inner and outer surfaces terminating at a lip,
said lip defining a needle mouth communicating with said central passageway;
at least a portion of said lip formed to create an edge.

2. The apparatus as recited in claim 1 wherein said edge is formed on said lip midway between said inner and outer surfaces.

3. The apparatus as recited in claim 1 wherein said edge is formed on said lip at and coextensive with said outer surface.
